(19) United States
(12) Patent Application Publication
(10) Pub. No.: US 2025/0387587 A1
(43) Pub. Date: Dec. 25, 2025

(54) BIOFEEDBACK USING BIO-RESONANT FREQUENCIES AND EMOTIONAL RESONANCE

(71) Applicant: Arrowin Li Ash, Vancouver, WA (US)

(72) Inventor: Arrowin Li Ash, Vancouver, WA (US)

(21) Appl. No.: 18/752,355

(22) Filed: Jun. 24, 2024

(51) Int. Cl. A61M 21/02; A61M 21/00; A61N 5/06

(52) U.S. Cl. CPC A61M 21/02; A61N 5/0622; A61M 2021/0011; A61M 2021/0022; A61M 2021/0027; A61M 2021/0044; A61M 2021/0055; A61M 2205/052; A61M 2205/057; A61M 2210/0693; A61M 2230/005; A61M 2230/10; A61N 2005/0659

(57) ABSTRACT

Methods providing biofeedback generally comprising: correlating an emotional state with an emotional resonance signal of a person; determining a bio-resonant frequency and wave form from a segment of the emotional resonance signal; and producing a feedback signal that is a higher harmonic of the emotional resonance signal. Said feedback signal can help adjust emotional states, balance brainwave activity, forge new neuro-circuits, increase interhemispheric communication, creativity and brain-efficiency thereby improving a full spectrum of nervous system activity from hyperarousal to deep sleep.

### FIELD OF THE INVENTION

The field of the invention is bio-feedback.

### BACKGROUND

The background description includes information that may be useful in understanding the present invention. It is not an admission that any of the information provided herein is prior art or relevant to the presently claimed invention, or that any publication specifically or implicitly referenced is prior art.

Brain science and research has found a correlation between spectrum of human mental and emotional states and the heart and brainwave frequencies being generated.

Sound and music have long been known to also affect spectrum of human mental and emotional mind states. Brain science and research has substantiated bioactivity of sound and its ability to provide therapeutic benefit to the receiver of that sound. Studies that correlate music with psychological effects shown that each key in the musical scale produces a different mental and emotional response in the brain and nervous system of the listener. This is been shown in research studies measuring stress response, psychological profile, and blood chemistry in response to sound tones presented in various keys. Depending on the individual, some keys will be uplifting and relaxing and other keys will create anxiety or feelings of depression. This is why music can make a person fall asleep, leap for joy . . . or cry.

Studies have shown that brainwave's respond dynamically, in different ways, to different types of music. Up till now though, no neuro-feedback methods have been able to bio accurately reflect the brain's own activity back to itself in the form of a feedback signal that represents a composition of the exact therapeutic sound and musical frequencies needed to balance the nervous system at any given moment of time.

This is given rise to the current invention whereby the audio feedback stream is a live real-time streaming bio accurate reflection of the brains own activity in the exact key and waveform shape that the brain is playing at the biological level. This provides the brain an additional level of information about itself not present in traditional forms of neurofeedback and allows the brain to be able to recognize its relationship to the qualitative changes in the neurofeedback signal and thereby more easily utilize the neurofeedback information to auto regulate its own processes. The brain recognizes the cause and effect nature of its own electrical activity much more clearly and strongly when the feedback is a mathematically accurate reflection of the brain's actual underlying electrical activity which has an exact waveform shape and exact frequency which will land somewhere on the musical spectrum. Since each key in the musical scale is capable of producing a different mental and emotional response, these bio accurate sounds allow the brain to learn what “key” it is playing in with its own brainwave activity. This allows the brain to neuro-associate the cause and effect relationship between one's mental and emotional state of mind and being and the qualitative nature of the “music” that the brain is producing. Over time the brain can learn how to change the tune of its own music in ways that affect brain and body chemistry as well as the functioning of the entire central nervous system. It is important to understand that simply corresponding a brainwave frequency with a musical key in the audio spectrum is not enough. Each key within the audio spectrum is a range of frequencies that fall within that key. A mathematically bio-accurate tone or sound must be the exact frequency within the range of frequencies that fall within that key in order to match the exact mathematical frequency of the underlying brainwave and be recognized by the brain and nervous system as such.

This invention utilizes that knowledge by multiplying the exact brainwave frequencies by octave-based mathematics to arrive at an audio signal with frequency or frequencies having the same characteristics as the original brainwave.

For example, the common practice of numerically adding a brainwave frequency to a preselected sound frequency to represent the brainwave frequency in the audio spectrum will most often produce an audio frequency that is not of the exact same musical key as the original wave. By multiplying according to our octave-based mathematical algorithm, we arrive at an audio frequency represents an exact frequency in the audio spectrum that matches the exact same musical key as the original brainwave.

This, in part, produces what we are calling bio-accurate sound. Bio accurate sound allows a whole new level of information density to be processed by the brain in an organized and meaningful fashion, in the language it understands, for increased speed of learning and reorganization of the neural structure for accelerated benefits in all the areas that neuro-feedback and sound therapies are known for.

Because harmonious tones sound good together, the brain begins to learn to “play” its own brain waves in keys that are more harmonious with each other across various bandwidths. This can allow a person to more easily access states of consciousness that depend on having brainwave activity in multiple bandwidths simultaneously. Because the brain doesn't like dissonance, it typically sticks to one dominant brainwave. If however, 2 bandwidths can exist harmonically in a non-destructive way, then they can coexist with equally strong amplitude. A lucid dream is an example of a non-ordinary state of consciousness that depends on the high amplitude activity of 2 distinct and distant brainwave bandwidths. Lucid dreams characterized by high amplitude theta together with high amplitude beta and/or gamma. Lucid dreaming is just one example of an extraordinary state of consciousness generated by the simultaneous presence of multiple brainwave bandwidths at strong amplitude. A number of other brainwave states can be combined for extraordinary meditation capacity and peak mental performance.

### Neural Noise Feedback

Neural noise appears in brainwave scans as a type of static composed of chaotic disorganized random electrical fluctuations occur in the electrical activity of the brain that show up as a form of static. This type of Neural noise obscures or interferes with efficient brain signaling and adds distortion to the underlying brain signals. Noisy brains are correlated with lack of focus, inability to concentrate, anxiety, age-related cognitive decline. This type of neural noise is also correlated with increased pain for those with fibromyalgia or other chronic nervous system conditions.

Brainwave training becomes naturally more efficient when the brain produces less neural noise and produces harmonious frequencies across the various bandwidths of its activity. Less neural noise means more efficient brain functioning across the broad-spectrum brainwave bandwidths. As the brain reorganizes its neural structure in response to the therapeutic sound feedback signal, the brain becomes more efficient at producing harmonious brain waves with the least amount of neural noise across the broad-spectrum brainwave bandwidths in mind states.

### Brainwave Variability (BWV)

However, there is another category of brain activity that is often mistakenly lumped into the category of Neural noise.

This “beneficial Neural noise” is actually a unique type of electrical activity of the brain, but it isn't actually noise at all. Unfortunately, most research software is programmed to analyze the brain waves for known frequency bandwidths at specific amplitudes and are prone to mis-characterize this unique electrical activity as “noise”. This led some early brain researchers to conclude that some Neural noise is necessary and beneficial for proper brain functioning.

However, brain science and research now recognizes that this beneficial type of “noise” is actually not noise at all. Leading brain researchers are now calling this important electrical activity in the brain “aperiodic brain signaling”. Aperiodic signaling is a measure of BWV (Brain Wave Variability).

With HRV, which is a measure of the harmonic content of the heart, the more variability in the heart, harmonics there is, the higher the correlation to mental and physical health and well-being bio-markers. Likewise with BWV, Brainwave variability is the capacity of the brain neurons to be equally efficient in any given moment of transmitting signals or inhibiting signals across a wide bandwidth of brainwave activity. Increased BWV is correlated with the ability to multitask, make split-second decisions or choices in a natural environment and the brain's capacity to shift from one mind state to another easily and quickly. In addition, BWV facilitates speed of learning as new neural networks that are formed in the presence of BWV will be more robust and able to adapt to changing demands in a dynamic environment.

The method of this an element within the audio stream providing the brain with dynamic positive feedback regarding the amount and type of brainwave activity falling into the category known as aperiodic signaling at any given moment.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### SUMMARY OF THE INVENTION

The inventive subject matter provides apparatus, systems and methods in which a biofeedback signal comprises of a multiplicity of bio accurate sounds, each of which is a dynamic ever-changing frequency that represents the exact mathematical frequency and waveform shape of the underlying brainwave or bio parameter being measured. These individual frequencies and sound structures, hereinafter called “elements”, are processed mathematically and blended together to create an ambient sound stream with therapeutic attributes due to the sound frequencies chosen. This unique feedback signal has the capacity to reflect not just some targeted brainwave or mind state as in previous neuro-feedback devices, but has the capacity to reflect all of the fine nuances, shapes and ripples of all of the brainwaves across the broad spectrum of brainwave activity and provide an information dense tapestry of sound to the brain.

In this type of biofeedback signal, all of the brain waves in each bandwidth brainwave activity are represented in a way that the brain can gain awareness of all of its activity simultaneously including its level of neural noise so that the brain can learn how reduce its level of neural noise and operating more efficiently across all bandwidths of brainwave activity. An important distinction that makes this type of feedback signal unique is that this type of feedback signal highlights the relationship between the various elements of its activity as opposed to simply providing audio or visual feedback when a brainwave is increasing or decreasing. The heart, brain and nervous system act as a holistic functioning whole and by providing feedback that encompasses the relationships between the parts of the nervous system, the entire brain and nervous system gain greater facility in learning and accessing mental and emotional states of being.

Furthermore, mathematical processing of the bio accurate audio elements produces a sound stream that not only reflects every aspect of the brain's activity in the brain's own language of frequency and mathematics, but the audio elements also represent the exact mathematically ideal therapeutic sound frequencies necessary for balancing the nervous system at any given moment of time.

Thus, this method of producing a neuro-feedback signal results in a feedback signal with dual function having the ideal properties of both an information dense, dynamically updating, real-time, mathematically bio-reflective feedback signal, as well as having the properties of a therapeutic sound treatment inasmuch as the feedback signal is composed of the mathematically precise sound tones and frequencies that facilitate brain and nervous system balance.

Bio-accurate sound. In some embodiments, biologically derived frequencies serve as exact mathematical reflections of the biological activity of the brain and heart while providing the mathematically correct therapeutic sound frequencies balance the respective brainwave activity.

Real-time streaming bio-accurate sound map. In some embodiments, the bio-accurate sounds are real-time streaming sounds which then are organized into a dynamic, real-time ambient sound stream having therapeutic properties which can be used for whole body, brain, heart and nervous system cooperative function and balance.

Neural noise feedback. In some embodiments an element within the audio stream provides feedback to teach the brain how to operate with low levels of neural noise and greater efficiency across the wide bandwidth of brainwave frequencies and mind states.

Sonic neuro-pacing. In some embodiments, elements within the feedback audio stream utilize biological frequency derived binaural beats and/or isochronic tones to lead the brain through the frequency spectrum and learn how to fluidly access a wide spectrum of mental and emotional activity.

Emotional wave-form feedback. In some embodiments an element within the feedback audio stream provides mathematically accurate feedback regarding the waveform shape of the heart or brainwaves it is generating and teaches the brain and nervous system the relationship between the emotions that we feel and the waveform shape it is generating. The scientific research and work of Dr. Manfred Climes demonstrated biological signals of the human body learn how to shift their waveform shape and that each of these waveform shapes fall into 5 mathematically definable ratios associated with each of 5 different emotions: anger, sadness, peace, joy and love.

Brainwave variability feedback. In some embodiments elements, an element within the audio stream provides the brain with dynamic positive feedback regarding the amount brainwave activity falling into the category known as aperiodic signaling at any given moment.

Various additional objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic of a person receiving a treatment in accordance with inventive concepts herein, including use of audio feedback.

FIG. 2 is similar to FIG. 1, except that the feedback generator produces photo-optic feedback, which is rendered through left and right images and/or pulses of light in a headset, pair of glasses or overhead mounted light pulse generator.

FIG. 3 is similar to FIG. 1, except that the feedback generator produces vibratory feedback, which is rendered through left and right vibrators placed on a person's wrists.

FIG. 4 is similar to FIG. 1, except that the feedback generator produces vibratory feedback, which is rendered through left and right sides, respectively, of a vibro-tactile table.

FIG. 5 is similar to FIG. 1, except that the feedback generator produces photo-optic feedback, which is rendered through left and right transcranial stimulators.

FIGS. 6A-6D show ratios that define waveforms that correlate with four emotions: (A) love; (B) hate; (C) anger; and (D) joy.

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Thus, there is still a need for improved methods of performing biofeedback.

### Bioactive Sound

Neuro active brain derived ambient music providing at least one element which is a mathematically bio accurate set of tones, pulses or frequencies.

Bio accurate sound refers to a sound whose waveform architecture is a mathematical reflection of the sub-audio brainwave waveform that it represents and whose frequency has an exact octave-based relationship to the underlying brainwave it represents. Used as a neurofeedback signal, a bio accurate sound mathematically encodes defined features of monitored brainwaves into the neuro-feedback signal before it is supplied as neurofeedback to the person receiving the neurofeedback treatment. Accordingly, methods are provided herein for mathematically encoding at least 1 brain waveform shape, frequency and amplitude, and sometimes also encoding coherence and relationship to other simultaneous measured waveforms. The information density of the neurofeedback signal as described herein, can be hundreds of times greater than a typical neurofeedback signal, and thereby can thereby provide the brain with a richer adaptive catalyst that increases neuro-plasticity, rapid brain learning and optimal shift towards peak performance.

Bio-accurate sounds are therapeutic in their own accord by virtue of 2 properties. 1) The bio accurate sounds and information rich form of dynamic feedback teaches the brain how to optimize and balance its functioning across a wide bandwidth of brainwave activity. 2) The bio-accurate sounds are also therapeutic by virtue of their ability to balance and strengthen brain and nervous system electrical activity in accord with the principles and methods outlined. The method producing the bio-accurate sound stream becomes further bioactive when the sub-audio bio accurate sound tone pulses are presented to the audio cortex in the form of a binaural pulse whose frequency is an exact mathematical sub-octave the bio accurate sound tones which compose the ambient music sound stream. This induces a state of coherence and symmetry between the 2 hemispheres of the brain. The value of this bioactive sound pulse is further enhanced as a form of feedback to the brain by providing said frequencies with a waveform architecture that exactly matches both the underlying sub harmonic heart and brain frequencies as well as the audio spectrum octaves which in part compose the ambient music therapeutic sound stream.

### Real-Time Streaming Bio-Accurate Sound Map

In practice, the method of this invention produces an ambient music sound stream composed of therapeutic sounds in the audio and sub audio spectrum. The composition of the ambient sound stream is a direct mathematical reflection of not just one brainwave frequency, whether it be the dominant brainwave frequency or a “fundamental frequency” or some predetermined ideal frequency, rather it is a direct reflection of all of the frequencies, rhythms and waveforms of the heart and brain's electrical activity, including some of its own “noise” which shows up in recording is a subtle background of pink or white noise composed of the feedback recipients own weak, stray or disorganized heart and brain wave harmonics. As the brain learns and gains the ability to let go of this electrical noise at the physiological level of its own functioning, the audio sound stream reflects this as the ever so slight background noise dissipates or disappears as an element within the ambient streaming sound.

When a person's brain activity is scattered chaotically into low-level electrical noise, then a person will feel stressed, unclear, confused and unfocused with no motivation. By giving the brain a “taste of its own noise” it learns how to not generate so much noise and instead intelligently operate in a more efficient fashion with less noise. As a person progresses with this type of bio accurate the ambient audio stream increases in quality, richness and complexity as the persons brain learns how to produce clear and coherent symmetrical heart and brain wave harmonics with little or no noise. With continued interaction with the ambient sound stream, the heart, brain and nervous system become a coherent symphony of therapeutic (- - - - - -) frequencies.

The ambient sound stream is not just a reflection of the brain waves but also their relationship to each other and/or their relationship to the harmonics of the heartbeat as determined by HRV. Thus the ambient sound stream not only provides the healing tones as per sound healing practice and theory, but because the resulting ambient music soundscape is a direct reflection of the holistic functioning and interplay of the body, brain and heart, the brain enters into a greater state of Neuro-plasticity wherein the brain learns how to achieve and maintain a broad spectrum of frequencies and patterns associated with various states of consciousness including the full range of mind states between hyperarousal and sleep. The breath, heart and brain all have interlinked rhythms that entrain each other into a single holistic expression of our total experience. By allowing this holistic interplay of biological rhythms to have its expression in the architecture of the ambient music, the entire nervous system, including the brain learns the most efficient processes for releasing stress and tension which shows up as “noise” or chaotic rhythms in a person's breath rate heart rate and brain waves. When the brain reduces or stops producing so much electrical noise, then the brain is more relaxed and has more energy available for clear productive and peak mind states.

The human brain has the ability to learn at the cellular level how to create new neural connections that support the brain's capacity to do something very easily once it has learned how to do it. Brain science calls this neuro-plasticity. Neuro-plasticity is what allows us to learn and remember a new skill. Just like riding a bicycle causes the brain to make new neural connections that encode new information about how to maintain balance while in the state of momentum. Once those neural connections have been created you can go 20 years without touching a bicycle and pick one up and ride it no problem. Likewise, when the brain learns how to let go of stress by letting go of the neuro “noise” that happens in the brain when its energy is scattered into weak and dis-harmonic patterns and frequencies, it will always have that ability because it forms new neural connections that make the higher functioning state the default mode of choice at the brain level.

The ambient music elements are tuned to harmonics the original biological frequency such that the resultant musical expression is a mathematically significant representation of the nervous system symphony. Each of the brainwave and/or heart beat parameters being measured have representation within the sonic tapestry of the ambient music soundscape. This gives the therapeutic ambient soundscape subtle information-rich nuances that the brain can use for deep learning in ways that other neuro-feedback signals cannot. Each bandwidth of the heart and brain harmonics are measured independently for amplitude, and waveform shape. This gives the brain a look at not just the dominant brainwave but all the nuances and textures representative of their heart brain and nervous system and what it looks like when all of those systems are working cooperatively and in coherence. Because of the richness of the sonic tapestry and its mathematical correlations to the brain and heart activity, it is extremely information dense. This information density creates greater neuro-plasticity and a deeper level of brain learning so the brain can learn to achieve and maintain a full spectrum of brain and mind states including focus, motivation, creativity, relaxation and the good deep sleep necessary to live a healthy naturally peak performance life.

### Neural Noise Feedback

The method of this invention is unique in that one of the elements within the audio feedback signal provides feedback to the brain as to the level of neural noise that it is producing. The method of this invention not only provides that basic information to the brain but also provides the brain with specifically which frequencies are producing the most neural noise and so that it can intelligently modify its frequency profile to produce less noise. The method of this invention accomplishes this by creating an element within the feedback signal with a frequency content that matches the precise spectral profile of the individual's neural noise at any given moment. In this way, precise information-rich feedback is provided to the brain about its own noise levels so that it can learn how to generate clear harmonic brainwave activity across a wide spectrum of brainwave frequencies and mind states with minimal amounts of neural noise. Furthermore, the audio frequencies provided as the feedback signal are formulated to provide either a bio-accurate frequency translation of the spectral emission of the individual's neural noise. Alternately, that feedback signal can be formulated as a frequency based “antidote” to the neural noise. This is accomplished much in the same way that noise cancellation headphones are able to cancel static background noise while still letting music or other people's voices come through. The feedback signal in this case becomes the inverse of the spectral emission of the neural noise or any other mathematical method to arrive at a frequency that reduces neural noise.

Applying the method of this invention results in a feedback signal that not only alerts the brain when it begins to produce neural noise, which is what the brain and nervous system needs to learn how to regulate its own activity, but the feedback audio signal itself consists of a unique frequency and waveform spectra having value in helping the brain and nervous system of a unique individual diminish its noise level. For these reasons, applying the method of this invention results in a feedback signal with dual function having the ideal properties of both an information dense, dynamically updating, real-time, mathematically bio-reflective feedback signal, as well as having the properties of a therapeutic sound treatment inasmuch as the feedback signal is composed of the mathematically precise sound tones and frequencies that facilitate brain and nervous system balance. This principle is applicable to the entire emission spectra of the brain and not limited to use with just the noise component.

### Sonic Neuro-Pacing

The method of this invention provides for an element within streaming audio for leading the brain via the process of entrainment to a frequency just above or below its current dominant frequency.

Mode 1: Mathematically altering one or both audio channels, or elements within each channel such that the frequency differential between one of the sound elements in each of the right and left channel exactly matches the brainwave frequency being measured plus or minus some small amount. This produces an intracranial pulse of sound at a frequency lower than the threshold of human hearing yet still “heard” as sound within the audio cortex. By adjusting this sound pulse within the audio cortex to be ever so slightly faster or slower than the actual brainwaves being measured, the brainwaves can be made to entrain and pace upward in frequency or downward in frequency The brain most easily entrains to a frequency so close where it's already at. The method of this invention facilitates easier entrainment towards a target frequency by allowing the frequency to “slide” naturally from where it's at into the slightly faster or slower entrainment frequency provided by the entrainment signal. The neuro-pacing function always keeps the entrainment signal ever so slightly ahead of the frequency being measured so that the frequency being measured keeps moving gracefully and naturally from one brainwave state to the next.

Mode 2: supplying an isochronic tone and or a percussive rhythm of an instrument or other natural or synthesized sound, timed or shaped to produce a continuous train of pulses with a pulse frequency equal to the brainwave frequency being measured, plus or minus some small amount, such that the beats per minute is ever so slightly faster or slower than the frequency being measured. This allows the brain to entrain slightly up or down in frequency. As the brain adjusts its own frequency pulse train also adjusts its frequency to always remain ever so slightly above or below the frequency being measured depending on whether the brain waves and nervous system need to shift upward or downward in order to achieve optimum nervous system balance in any measured bandwidth.

Mode 3: With or without the use of headphones, Monaural Beats can be used when the differing tones are supplied through speakers. Alternately, and in addition, all 3 modes described above can be used simultaneously to transmit their sub-audio frequencies and pulses via the use of variable intensity light, pulsed light, and or vibrotactile transmission tables recliners and other such devices.

Any of the 3, or all of the above 3 modes can be used together simultaneously.

### Emotional Wave-Form Feedback

Manfred Clines, a biorhythm researcher studied the waveforms heart and brain discovered there is a characteristic waveform shape that encodes the emotional content of the sound wave or frequency. He discovered 5 characteristic waveforms which correlated with 5 basic emotions. These waveform shapes produced by our heart and brain waves shift in advance of our physical awareness and experience of these emotions.

Our unique and proprietary algorithm allows the brain and nervous system to become aware of what it's doing moments before an emotion arises or shift in mind state occurs.

This information in conjunction with the other elements of the bio-accurate streaming sound map provides brain and nervous system with the information it needs to understand the relationship between mind and emotions and increases our brain's capacity to manage emotional states without our conscious intervention.

People with stuck emotions will find that their brain learns how to unstick itself simply by becoming aware of the mental perturbations and noise that get in the way.

A healthy brain is capable of expressing a full range of human emotion as needed, but a disorganized brain full of chaotic brainwave patterns is less able to access positive emotional states like peace, joy, love and bliss. Each of these positive emotional states has a characteristic signature waveform shape that can be trained for. As the brain, heart and nervous system learn the waveforms associated with each emotion the person gains greater capacity to feel and experience these emotions.

Furthermore, in one mode of operation, “waveform entrainment” is used to entrain the wave shape of a person's heart and brain waves into the desired waveform shape (which can be expressed at any frequency). Waveform pacing is accomplished by the method of this invention programmed into software by the processing the frequencies within the ambient soundscape such that elements within the bio accurate streaming sound map are algorithmically shifted to match one of the 5 waveforms and the corresponding emotion that the brain is learning to work with. As the heart and/or brainwaves shift towards the chosen waveform, the algorithm provides positive feedback in the form of audio or visual reward as the brainwave moves toward the desired waveform until it is achieved.

In another mode of operation and method of achieving waveform pacing is that certain audio elements present in the background the ambient soundscape or some other audio element dissipates progressively as the person's heart, brain and nervous system harmonize into the desired emotional waveform.

Any number of vibratory inputs or tones can be used to alert the nervous system when it's predominant heart and/or brain waveforms are shifting towards a waveform that allows a specific emotion to flow. Other vibratory inputs that can be successfully used for this purpose include variable intensity light, colored light, pulsed light and vibrotactile stimulation.

### Brainwave Variability (BWV) Feedback

Brainwave variability feedback can help the brain more easily learn how to shift its rhythms up and down the frequency spectrum and move fluidly from one mind state to another. This prevents the brain from becoming stuck in any one frequency pattern through habituation of thinking patterns and lifestyle habits. Being able to achieve a clear pleasant hyperarousal with its associated focus, concentration and motivation is important, but it is even more important to be able to shift fluidly between desired states. Shifting from hyperarousal to deep sleep can be a long and sometimes impossible process for a brain not able to shift easily from one state to another. The ability to shift from relaxation to hyperarousal, focus and motivation, then back to deeply relaxed states of peace, meditative awareness, dreaming and deep rejuvenating sleep again is potentially more valuable than typical neuro-feedback training the brain to simply achieve some predetermined set of frequencies. As the brain learns how to move fluidly from one state to another, new neural connections are created. As new connections are used, they are strengthened to the point that they become more or less permanent and will always be there to help maintain brain and heart rhythms of optimum nervous system balance regardless of what brainwave mind state bandwidth we are in.

The method of this invention creates an element within the audio stream which provides the brain with dynamic positive feedback regarding the amount and type of brainwave activity falling into the category known as aperiodic signaling at any given moment.

This is accomplished through the mathematics of the golden ratio. The golden ratio is maximally aperiodic. By providing a feedback signal that signals the brain when the ratios of the various brain waves and heart waves relate to each other by the golden mean and it's harmonics, a proxy for aperiodic brainwave activity can be had and a feedback signal can be created that allows the brain to achieve positive feedback when maximally aperiodic activity is being exhibited.

The golden mean is a ratio defined as 1.618 . . . , Therefore, if a person had a dominant brainwave in the theta bandwidth at exactly 5 cps then, we would multiply that frequency times 1.6182 arrive at the first golden mean harmonic which is 8.09. Each resultant frequency is multiplied times the golden mean to arrive at the following frequencies 8.09, 13.89, 21.18, 34.27, 55.45. Spikes in brainwave activity above a given noise threshold and when brainwave activity is exhibited in the vicinity of the following frequencies: 8.09, 13.89, 21.18, 34.27, 55.45, then that activity will be categorized as aperiodic activity that represents an increase in brainwave variability. Secondary and tertiary aperiodic activity points can be monitored which are the frequencies that result from multiplying the dominant brainwave frequency by the golden mean ½ and ¼ harmonics.

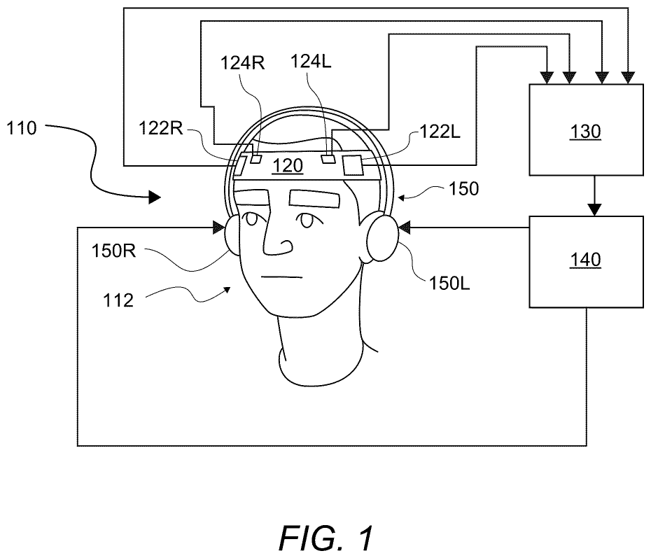

FIG. 1 generally depicts a person 110 receiving a treatment in accordance with inventive concepts herein. A headband 120 positions sensors 122L and 122R about the temporal regions of the person's head 112, and sensors 124L and 124R on opposites sides of the forehead. Signals from the sensors are carried by wires to a computing device 130, which analyzes the sensor-derived signals, and directs a feedback generator 140 to produce audible and/or sub-audible feedback, which is rendered through headphones 150 having left 150L and right 150R sides.

The computing device 130 should be considered generically to include laptops. The headphones 150 should be considered generically to include a left speaker 150A and a right speaker 150B, whether for example in an over the car headphone configuration, an earbud configuration, or any other suitable configuration.

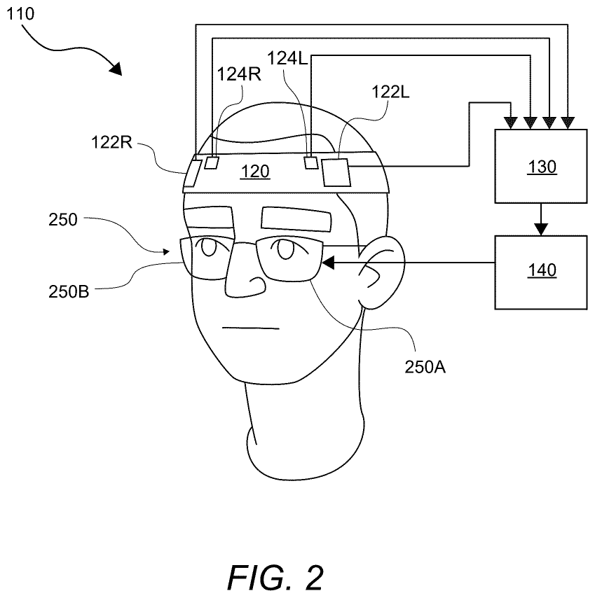

FIG. 2 is similar to FIG. 1, except that the feedback generator 140 produces photo-optic feedback, which is rendered through images produce in a headset 250. The headset 250 should be considered generically to include a left display 250A and a right display 250B, whether for example in a headset of the type used for virtual reality sessions, eyeglasses, contact lens type displays, intra-ocular displays, or any other suitable displays.

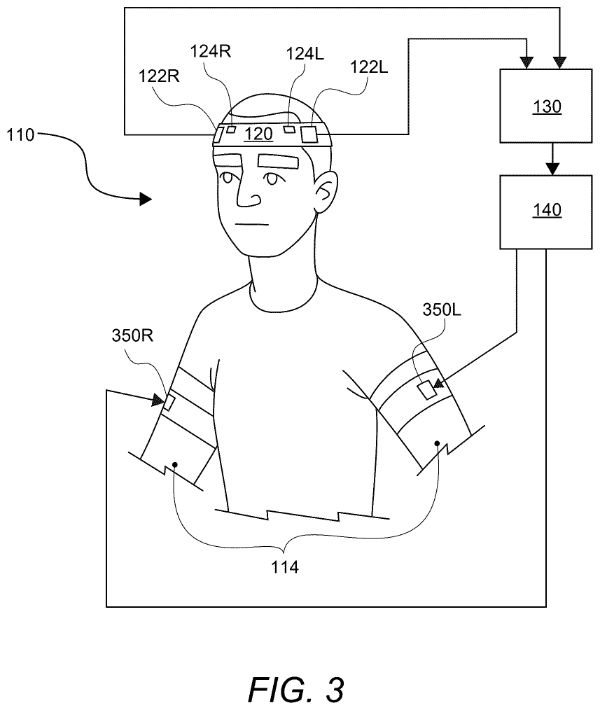

FIG. 3 is similar to FIG. 1, except that the feedback generator 140 produces vibratory feedback, which is rendered through left and right vibrators 350L, 350R placed on the persons' wrists 114. Positioning of the left and right vibrators 350L, 350R should be considered generically, with all additional or alternative, anatomically reasonable vibrator positions also contemplated.

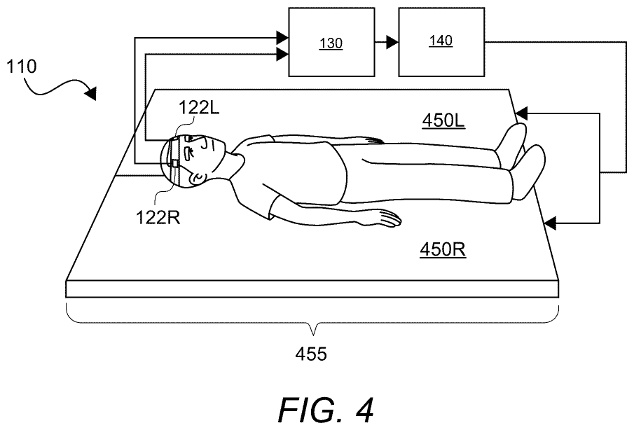

FIG. 4 is similar to FIG. 1, except that the feedback generator 140 produces vibratory feedback, which is rendered through one or more vibrators 450L, 450R on the left and right sides, respectively, of a vibro-tactile table 455.

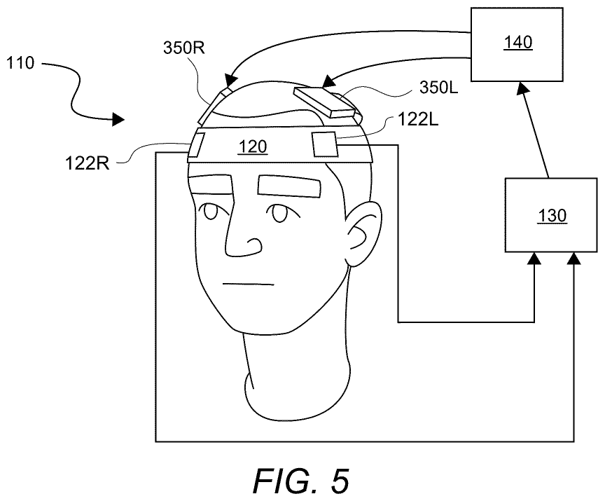

FIG. 5 is similar to FIG. 1, except that the feedback generator 140 produces vibratory feedback, which is rendered through left and right transcranial stimulators 550L, 550R. It is contemplated that transcranial feedback signals can be delivered via infrared or red LED, by laser, or by any other suitable delivery mechanism.

In FIGS. 1-5, there are left and right brain signals, and left and right feedback signals. It is contemplated that in each of those cases, the left and right feedback signals can be rendered to the person 110 in a combined manner or independently. Where the left and right feedback signals are independent, the feedback signals can be rendered on the same or opposite side of the body as the source of the brain signals.

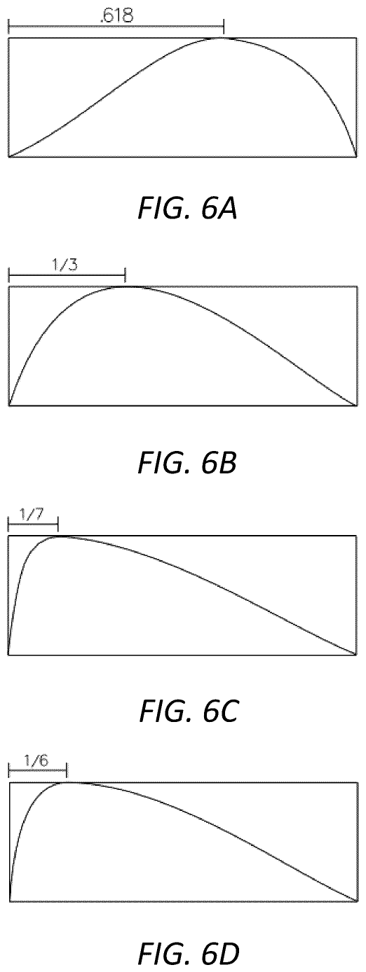

FIGS. 6A-6D show the ratios that define waveforms that correlate with four emotions: (A) love; (B) hate; (C) anger; and (D) joy.

The inventive subject matter includes two primary breakthroughs applied to neurofeedback: 1) the use of bio-resonant a.k.a. bioactive sound, light and vibration frequencies, and 2) the ability to use the voice and or electroencephalogram (EEG) to detect and measure emotional resonance in the consciousness of the user.

Bioactive neurofeedback signal: This is a neurofeedback signal composed of bio resonant sound, light and vibration frequencies. As used herein, a bio-resonant frequency is a frequency that is resonant with a frequency emitted by a human brain. As used herein, the feedback frequency is either a human-perceptible harmonic of bio resonant frequency, or a sub-perceptible bio-resonant frequency having measurable effect on brain waves or other biological parameters of measurement like HRV, GSR etc. For example, the same bio resonant frequency set introduced through a perceptible medium like tactile vibration can also be introduced through biologically active, yet not physically perceptible mediums. Sub-perceptible Mediums like pulsed magnetic fields, amplitude pulse-modulated radio waves, ultrasound, infrared or far-infrared light penetration into the brain tissue itself at the areas of the temple where the skull is thin or nonexistent, etc. Tuning a feedback frequency to a bio-resonant frequency allows resonance to occur between the light sound and vibration, or other feedback frequencies of the electromagnetic spectrum and the brainwaves that created them. Resonance allows a transfer of energy and information to the brain, nervous system and entire body. Bio-resonant frequencies facilitate up regulation of the brain and nervous system as those previously weak or missing frequencies are integrated begin to auto-generate.

Emotional Resonance Feedback: This is a method of detecting and categorizing emotional states encoded in the waveform of the EEG brainwave being measured. “Waveform” refers to the architecture of the wave itself irrespective of frequency. Sinewave is very different than a sawtooth wave or square wave. The wave can have a slow incline with the late peak in a sharp decline, or conversely, they can have a sharp incline with an early peak and the slow decline, which can be used to develop more self-awareness and emotional/social intelligence while developing the nervous system memory for accessing higher emotional states like compassion, joy and peace. Neurofeedback technology with emotional resonance signal feedback is capable of accurately identifying and providing real-time feedback on a variety of prime emotions like love, compassion, joy, gratitude, anger, etc. Likewise, it can modulate the architecture of any of the feedback and/or entrainment frequencies to match the signature of any emotion like love, compassion or joy. As used herein, an emotional resonance signal is an electrical signal originating from human brain activity and is associated with an emotional state.

Identifying and correlating the emotional states of EEG signals can include observing EEG signals after exposing the patient to an emotional stimulant and correlating wave forms of the EEG signal with different emotional states.

EEG/HRV Modulated Multisensory Entrainment: Entrainment refers to the brain's ability to tune itself to rhythms and frequencies in the external environment. This is how and why music can have such powerful mental and emotional effects that people can feel it in their body. Music has the ability to entrain emotional states because the music creates a shift in the brainwaves and nervous systems of the listeners. The neurofeedback technology described herein evokes the entrainment principle by providing the brain with a multisensory entrainment field consisting of bio-resonant frequencies of light, sound and physical vibration. This bio-resonant entrainment field can then guide the user's brainwave profile from where it is at, like the end of a hectic day at work, to the idealized brainwave profile and associated mind state preferred, like or ultra-deep relaxation or meditation for example. Any number of mind states can be targeted with entrainment frequencies.

Entrainment is used as a method of teaching the brain how to experience a particular brainwave profile and associated mind state. Entrainment works best when the user simply relaxes and surrenders to the experience which allows the users brainwaves to begin tuning themselves to the entrainment frequencies. This is like training wheels for the brain before the brain learns how to produce the frequencies on its own.

The inventive neurofeedback technology intelligently defines the ideal entrainment frequencies for each unique individual based on a system analysis of their EEG harmonics.

By using bio-resonant entrainment fields precisely tuned to the person's own brainwaves, the neurofeedback technology can balance and strengthen the users nervous system while increasing the brains neural recognition and receptivity to the signal and its adaptive response towards the entrainment goal.

It is contemplated that a multisensory entrainment session can last anywhere from a few minutes to an hour. The sessions can be simple or they can be composed with many layers of different musical and artistic styles. The user can also add entrainment into their neurofeedback sessions to help the brain learn how to optimize the neural connections responsible for the desired brainwaves and mind state.

Group Feedback and Entrainment: It is also contemplated that sessions can be saved as templates and top favorites can be shared amongst users in categories like peak-performance entrainments, meditation entrainments, creative inspiration entrainments, etc. The benefits of entrainment are multiplied by the number of people together.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of providing feedback to a person, comprising:
correlating an emotional state with an emotional resonance signal of the person by algorithmically matching an architecture of a wave form of the emotional resonance signal with an architecture of a wave form of the emotional state;
determining a bio-resonant frequency and wave form from a segment of the emotional resonance signal;
producing a feedback signal comprising a reproduction of the wave form of the segment at a higher harmonic of the frequency; and
rendering the feedback signal to the person.

2. The method of claim 1, further comprising deriving the emotional resonance signal from an electroencephalogram (EEG) of the person.

3. The method of claim 1, wherein the emotional state is one of love, hate, anger, and joy.

4. The method of claim 1, wherein the bio-resonant frequency is human-perceptible light.

5. The method of claim 1, wherein the bio-resonant frequency is human-perceptible sound.

6. The method of claim 1, wherein the bio-resonant frequency is human-perceptible tactile vibration.

7. The method of claim 1, wherein the bio-resonant frequency is delivered via sub-perceptible frequency consisting of pulsed magnetic fields.

8. The method of claim 1, wherein the bio-resonant frequency is delivered via sub-perceptible frequency consisting of amplitude pulse-modulated radio waves.

9. The method of claim 1, wherein the bio-resonant frequency is delivered via sub-perceptible frequency consisting of pulse-modulated infrared or far-infrared light penetration of the brain or other nervous system tissue.

10. The method of claim 1, further comprising the step of modulating the feedback signal to match a different emotional state.

11. The method of claim 1, wherein a shifting amplitude of the feedback signal corresponds with a shifting amplitude within the segment.

12. The method of claim 1, wherein the determined frequency comprises a dominant frequency within the segment.

13. The method of claim 1, wherein the determined frequency comprises an instantaneously dominant frequency within the segment.

14. The method of claim 1, wherein the higher harmonic is an octave higher than the determined frequency.

15. The method of claim 1, wherein the higher harmonic is a musical harmonic other than a higher octave of the determined frequency.

16. The method of claim 1, wherein the step of introducing the feedback signal to the person is accomplished at no more than 300 msec delay from detecting the emotional resonance signal.

17. The method of claim 1, wherein the step of introducing the feedback signal to the person is accomplished at no more than 50 msec delay from detecting the emotional resonance signal.

18. The method of claim 1, wherein the step of introducing the feedback signal to the person lasts for at least 10 cumulative minutes within a 60 minute period.

19. The method of claim 1, further comprising rendering a second signal to the person as a superimposition upon the rendered feedback signal.

20. The method of claim 15, wherein the second signal is consciously detectable by the person during rendering of the feedback signal.

21. The method of claim 15, wherein the second signal is not consciously detectable by the person during rendering of the feedback signal.

22. The method of claim 1, further comprising rendering the feedback signal to balance the person's emotional resonance signal.

23. The method of claim 1, further comprising rendering the feedback signal to improve the person's depth of sleep.
